(19) United States
(12) Patent Application Publication
(10) Pub. No.: US 2004/0267245 A1
(43) Pub. Date: Dec. 30, 2004

(54) Cautery holder

(76) Inventors: Phillip M. Riney, Owensboro, KY (US); Ross W. Cotton, Owensboro, KY (US)

Correspondence Address: BOWERS HARRISON LLP, GARY K. PRICE, ESP., 25 RIVERSIDE DRIVE, PO BOX 1287, EVANSVILLE, IN, 47706-1287, US

(21) Appl. No.: 10/877,143

(22) Filed: Jun. 25, 2004

Related U.S. Application Data
(60) Provisional application No. 60/480,885, filed on Jun. 25, 2003.

(51) Int. Cl. A61B 17/00

(52) U.S. Cl. 606/1

(57) ABSTRACT

A holder for securing surgical instruments and equipment during surgery. The holder includes a surface portion attached to a base portion. The base portion includes an adhesive layer and a protective sheet disposed over the adhesive surface. The surface portion having a first section that is used as a securing area for receiving, securing and cleaning the cautery, and a second section that is further used as a cleaning area for the cautery. The first section is constructed of a material that cleans the pointed portion of the instrument as the pointed portion penetrates and is removed from the first section. The second section preferably defines a V-shaped configuration, and is used for directly scraping off debris from the pointed portion of the instrument.

### CROSS REFERENCES TO RELATED APPLICATIONS

U.S. Provisional Application No. 60/480,885, filed Jun. 25, 2003, with title “Bovie Cautery Holder” which is hereby incorporated by reference. Applicant claims priority pursuant to 35 U.S.C. Par. 119(e)(i).

### STATEMENT AS TO RIGHTS TO INVENTIONS MADE UNDER FEDERALLY SPONSORED REASEARCH AND DEVELOPMENT

Not Applicable

### BACKGROUND OF THE INVENTION

1. Field of the Invention

This invention relates to a holder for securing surgical instruments and equipment during surgery and, more particularly, to a cautery holder that secures or retains the surgical instrument and may be releasably attached to either the drape or the patient, usually in the region of the surgical site.

2. Brief Description of Prior Art

Surgical procedures require the use of various types of instruments and equipment. As referred to herein, the term “surgical instrument” will refer specifically to cauterizers, and the like, commonly known as the BOVIE cautery.

It is necessary that the surgical instrument be available close to the surgical site during the medical operation. Desirably, the surgical instrument would be attached to the patient's body, or to drapes or covers shielding the patient's body, in order to minimize inconvenience to the surgeon. Unfortunately, due to uneven surfaces afforded by the patient's body, it has been impractical for the surgical instrument to be any closer than a nearby surgical tray.

The use of surgical tool holders and holding pouches are known in the art. More specifically, such pouches heretofore devised and utilized for the purpose of storing and retaining various surgical tools during periods of non-use are known to consist basically of familiar, expected and obvious structural configurations, notwithstanding the myriad of designs encompassed by the crowded art which have been developed for the fulfillment of countless objectives and requirements.

More particular, the surgical tool pouches and holders presently available in the art are substantially limited in both concept and function inasmuch as they are designed primarily as a tool storage means. The pouches are often constructed of materials which are rugged and cumbersome inasmuch as such materials add to the durability of the pouches per se, and little or no thought has been given to the idea of utilizing the pouches in an operating environment, such as during a medical operation. Further, the inventor is unaware of a prior art pouch or holder designed to hold a surgical instrument having the physical characteristics of a BOVIE cautery, that can be utilized during a medical operation.

Therefore, it can be appreciated that there exists a continuing need for a new and improved surgical instrument holder designed for use with a BOVIE cautery, that can be utilized in an operating environment. As will be seen from the subsequent description, the preferred embodiment of the present invention overcome limitations of existing surgical tool holders for use with cautery tools and the like.

### SUMMARY OF THE INVENTION

In view of the disadvantages inherent in the known types of surgical tool pouches now present in the prior art, the present invention provides an improved surgical instrument holder construction wherein the same can be used by the medical profession in an operating room environment. As such, the general purpose of the present invention, which will be described subsequently in greater detail, is to provide a new and improved holder designed to hold a surgical instrument having the physical characteristics of a cautery, that can be utilized during a medical operation.

To attain this, the present invention includes a surface portion attached to a base portion that is a substantially flat surface. The base portion includes an adhesive layer and a protective sheet disposed over the adhesive surface.

The surface portion defines a first section that is used as a securing area for receiving, securing and cleaning the cautery, and a second section that is further used as a cleaning area for the cautery. In application the first section receives the pointed portion of the cautery instrument and maintains the cautery in an upright position for further access and use. The first section is constructed of a material that cleans the pointed portion as the pointed portion penetrates and is removed from the first section.

The second section preferably defines a V-shaped configuration, and is used for directly rubbing or scraping off debris from the pointed portion of the cautery.

### BRIEF DESCRIPTION OF THE DRAWINGS

FIG. 1 is a top perspective view of the preferred embodiment of the present invention, a cautery holder.

FIG. 2 is a side view of the cautery holder of FIG. 1.

FIG. 3 is a sectional view of the cautery holder of FIG. 2.

### DESCRIPTION OF THE PREFERRED EMBODIMENT

During a typical surgical procedure, disposable drapes cover those portions of the patient's body that are not involved directly in the surgical procedure. Once the patient has been draped and the surgical site on the patient is exposed, the physician may use a cautery to cauterize various body tissues. The surgical instrument is frequently moved back and forth between a surgical tray nearby the patient and the surgical site that is being addressed. The current invention relates to a cautery holder that secures or retains the surgical instrument and may be releasably attached to either the drape or the patient, usually in the region of the surgical site.

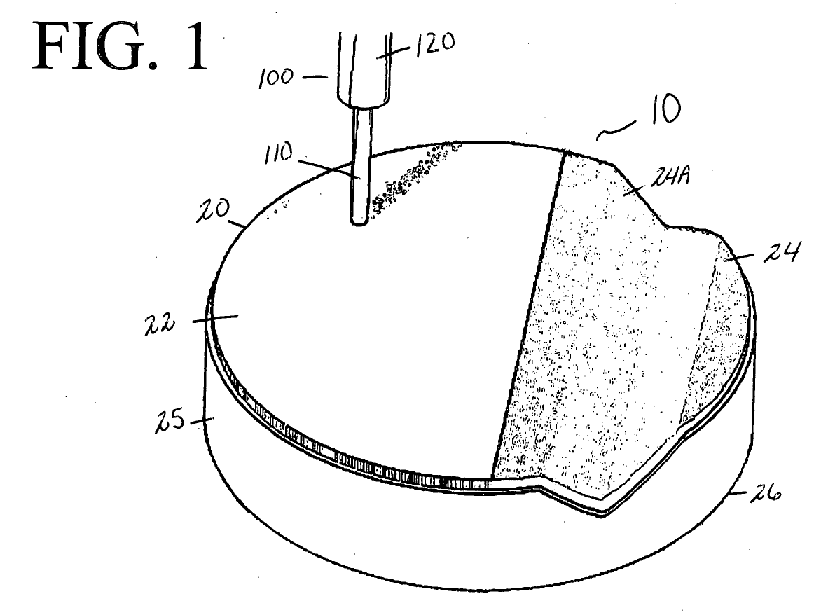

FIGS. 1-3 illustrate the preferred embodiment of a cautery holder 10 made in accordance with the present invention. The cautery holder 10 designed to hold a surgical instrument having the physical characteristics of cauterizers and the like, and as will be further described can be utilized during a medical operation.

FIG. 1 illustrates the cautery holder 10 securing a prior art cautery 100. Since the prior art cautery 100 is not necessarily apart of the present invention, it will not be required to describe fully its physical characteristics or its medical function. For purposes herein, the cautery 100 generally includes a pointed portion 110, and a handle portion 120, said pointed portion 110 being in contact with the patient during the act of cauterizing.

As shown in the drawings, the cautery holder 10 generally comprises a surface portion 20 and a base portion 25. The surface and base portions 20, 25 preferably having a substantially circular configuration.

The surface portion 20 defining a first section 22 that as will be discussed serves as a securing area for receiving and securing the cautery 100, and a second section 24 that as will be discussed serves as a cleaning area for the cautery 100.

First Section 22—Securing Area:

As shown, the first section 22 preferably having a generally half-moon configuration however, other configurations having the embodiments described is understood. In application the first section 22 serves two purposes: first, the first section 22 receives the pointed portion 110 of the cautery 100 and maintains the cautery 100 in an upright position (as shown in FIG. 1) for further access and use, second, the first section 22 is constructed of a material that cleans the pointed portion 110 as the pointed portion 110 penetrates the first section 22.

The characteristics of the preferred material of construction of the first section 22 is as follows:

the material must be a non-melting and non-flammable material since the pointed portion 110 of the cautery 100 can be hot when it penetrates the first section 22,

the pointed portion 110 of the cautery 100 should penetrate the material easily during application,

the material should have sufficient density to maintain the cautery 100 in an upright position once the pointed portion 110 of the cautery is received within the first section 22,

the material must be sterilized,

no particles can be present in the material that can contaminate or transfer to the pointed portion 110 of the cautery 100, and

preferably, the material is somewhat abrasive in order to clean debris from the pointed portion 110 of the cautery 100 as it is received and removed from the first section 22.

The first section 22 can be constructed of a non-woven fiber material having a loose, random attachment between the fibers. For example, there is known a stainless steel wire construction similar to a “scrub pad” having wires woven or knit into a selected shape. Alternatively, there is known material having random wire that is fused (sintered) at random contacts within the wire bundle to create a non-woven mass of fibers that retain its external configuration and some strength internally.

An alternate material can be a thermoset plastic fiber such as polymide, epoxy or thermoset polester that will not melt and is flame resistant. For example, there is known a random direction fiber pad that is bonded in a loose mass similar to a SCOTCH BRIGHT pad available on the market.

Another alternate material can be an abrasive particle filled soft material such as silicone rubber. The abrasive can be aluminum oxide particles for example, that are mixed by 20-40% volume percent into a soft silicone rubber. This can be molded into a pad having vertical slots or cuts, or a hole pattern such that the pointed portion 110 of the cautery 100 could penetrate at the locations of the hole patterns. The abrasive particles in the silicone rubber cleans the pointed portion 110 as it is received and removed from the first section 22.

Second Section 24—Cleaning Area:

As shown, the second section 24 preferably defining a V-shaped configuration however, other configurations having the embodiments described is understood. In application the V-shaped second section 24 includes a backing surface 24A and is used for directly rubbing or scraping off debris from the pointed portion 110 of the cautery 100.

The characteristics of the preferred material of construction of the second section 24 is as follows:

the material must be a non-melting and non-flammable material since the pointed portion 110 of the cautery 100 can be hot when in contact with the second section 24,

the material must be sterilized,

the material must be sufficiently abrasive in order to cause any debris on the pointed portion 110 to flake off the pointed portion 110 as the pointed portion 110 contacts the second section 24;

no particles can be present in the material that can contaminate or transfer to the pointed portion 110 of the cautery 100, and

the surface of the material must be resilient so that it can deform and adapt to the contour of differently shaped pointed portions 110.

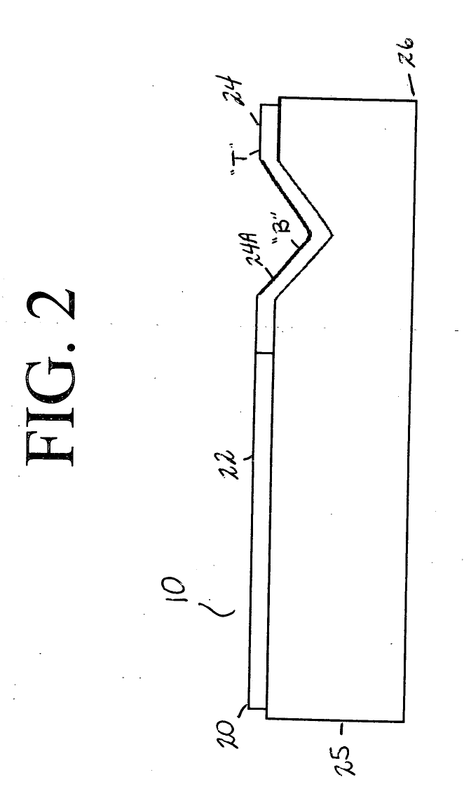

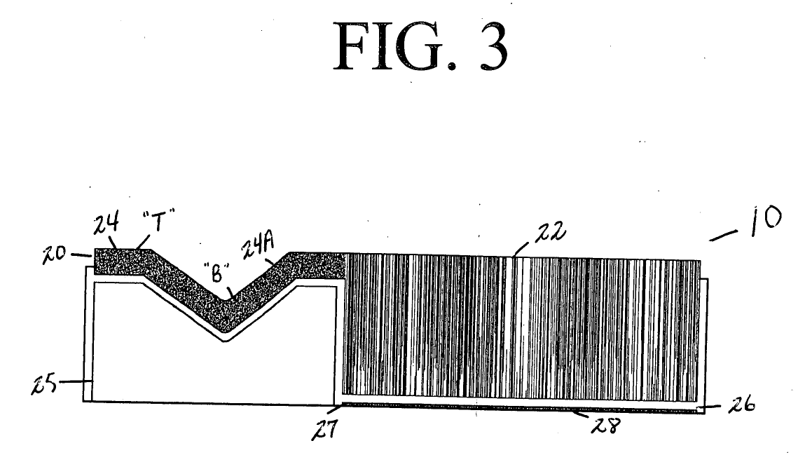

In the preferred embodiment, the thickness of the material of the second section 24 may vary. For example, the fibers at the bottom section (displayed as “B” in FIGS. 2 and 3), can be shorter and stiffer to enable scrapping off hard to remove debris from the pointed portion 110 of the cautery 100; and, the fibers at the top section (displayed as “T” in FIGS. 2 and 3), can be softer for less hardened debris in order to enable the entire pointed portion 110 to be pressed into the fibers for cleaning.

The second section 24 can be constructed of a material having a woven fiber layer (bottom section) bonded to a soft polyurethane foam backing (top section). The woven fiber layer can be constructed of nylon, polyester or other common fabric. Abrasive particles such as aluminum oxide or silicone carbide would be bonded to it. This abrasive layer on the fabric would then be coated with a thin bonding material to prevent particles coming off the material.

An alternate material for the bottom section of the second section 24 can be a needle punched stainless steel fiber through a fiber backing such that the stainless steel fibers are all standing on end. The fibers would be closely packed like a carpet such that the ends of the fiber would all stand up. As such, the pointed portion 110 of the cautery 100 is cleaned as the pointed portion 110 is in frictional contact with the fiber ends. Edges of the pointed portion 110 can be pressed into the fiber bundle to further assist in cleaning edges and non-uniform areas of the pointed portion 100.

The second section 24 is suitable for cleaning the cautery 100 during use, and in particular, will enable the surgeon to easily and quickly clean hardened debris from the pointed portion 110 of the surgical instrument 100.

The surface portion 20 as described above is appropriately affixed to the base portion 25. The base portion 25 having a substantially flat outer surface 26. The base portion 25 further includes an adhesive surface 27 (shown in FIG. 3) on its outer surface 26. The adhesive surface 27 is protected by a removable protective sheet 28 disposed over the adhesive surface 27. As will be understood, the base portion 25 is of sufficient rigidity to prevent the pointed portion 110 of the instrument 100 from penetrating during use.

If desired, the first section 22 and/or second section 24 of the surface portion 20 can be colored in order to increase the visibility of the holder 10.

During typical use, the surgeon will repeatedly insert the pointed portion 110 of the surgical instrument 100 within the first section 22 of the surface portion 20 for storing, and remove the instrument 100 for use. The sufficiently abrasive material of the first section 22 will enable the surgeon to clean debris from the pointed portion 110 each time the surgical instrument 100 is inserted or removed from the holder 10. As such, the first section 22 of the surface portion 20 forms a receptacle that permits the surgical instrument 100 to be withdrawn and reinserted. Moreover, the abrasive material of the first section 22 will enable the surgeon to easily and quickly clean debris from the instrument 100. It is critical that the material of the base portion 25 is of sufficient rigidity to prevent the pointed portion 110 of the instrument 100 from penetrating the base portion 25 during use.

As described, the abrasive material of the second section 24 will enable the surgeon to clean hard to remove debris from the pointed portion 110 of the instrument 100. As such, the V-shaped second section 24 forms a means to permit the pointed portion 110 of the surgical 100 to be scrapped or frictionally dragged across the second section 24 at various angles in order to remove the debris from the instrument 100.

In application, the holder 10 can be attached to the surgical drape or a portion of the patient's body near the site of the operation. Attachment of the holder 10 is accomplished by removing the protective sheet 28 as described above and contacting the adhesive surface 27 to the drape or the patient.

Once the holder 10 is in place, the surgeon can secure the surgical instrument 100 within the first section 22 of the holder 10. This is done simply by the surgeon grasping the surgical instrument 100 by the handle portion 120 and directing the pointed portion 110 within the first section 22. Once the pointed portion 110 of the instrument 100 is inserted, the first section 22 retains the surgical instrument 100 in an upright position for further access and use. The surgeon can quickly and easily remove and reinsert the instrument 100 throughout the duration of the operation.

As previously described, the first and second sections 22, 24 of the surface portion 20 can be used to clean the pointed portion 110 of the surgical instrument 100 during the course of the operation. Once the operation has been completed, the surgical instrument holder 10 can be disposed along with the surgical drapes.

As will be apparent from the foregoing description, the holder 10 according to the invention provides a number of advantages for securing surgical instruments having the physical characteristics of cauterizers and the like. The holder 10 can be attached easily to a patient's body or surgical drape. The abrasive material of the first and second sections 22, 24 of the surface portion 20 will enable the surgeon to easily and quickly clean debris from the surgical instrument 100 during the operation. Moreover, if the sections of the surface portion 20 has been colored with a dye, the holder 10 can be more easily located during surgery. Further, because the holder 10 will be inexpensive to manufacture, the holder 10 can be discarded at the end of the operation, together with the surgical drape.

Although the description above contains many specificities, they should not be construed as limiting the scope of the invention but as merely providing illustrations of some of the presently preferred embodiments of this invention.

It will be obvious to those skilled in the art that modifications may be made to the embodiments described above without departing from the scope of the present invention. Thus the scope of the invention should be determined by the appended claims in the formal application and their legal equivalents, rather than by the examples given.

I claim:

1. A surgical instrument holder comprising:
a surface portion attached to a base portion,
wherein the surface portion comprising a first section and a second section,
wherein the first section comprising a first material and the second section comprising a second material.

2. The surgical instrument holder as recited in claim 1, wherein the second section having a V-shaped configuration.

3. The surgical instrument as recited in claim 2, wherein the second section further including a backing surface.

4. The surgical instrument as recited in claim 3, wherein the backing surface comprising a third material.

5. The surgical instrument as recited in claim 1, wherein the first material having sufficient density to maintain a cautery instrument in an upright position when a pointed portion of the cautery is received within the first section.

6. The surgical instrument as recited in claim 1, wherein the base portion includes a substantially flat outer surface.

7. The surgical instrument as recited in claim 6, wherein the flat outer surface is an adhesive surface.

8. The surgical instrument as recited in claim 7, wherein a removable protective sheet covers the adhesive surface.

9. The surgical instrument as recited in claim 1, wherein the first material is less abrasive than the second material.

10. The surgical instrument as recited in claim 4, wherein the first material and the second material are less abrasive than the third material.

11. A surgical instrument holder comprising:
a surface portion attached to a base portion, said base portion including an attaching means to temporarily attach the surgical instrument holder to a patient,
wherein the surface portion comprising a first section and a second section,
wherein the first section comprising a first material and the second section comprising a second material.

12. The surgical instrument holder as recited in claim 11, wherein the second section having a V-shaped configuration.

13. The surgical instrument as recited in claim 12, wherein the second section further including a backing surface, said backing surface comprising a third material.

14. The surgical instrument as recited in claim 11, wherein the attaching means is an adhesive surface.

15. The surgical instrument as recited in claim 14, wherein a removable protective sheet covers the adhesive surface.

16. The surgical instrument as recited in claim 11, wherein the first material is less abrasive than the second material.

17. The surgical instrument as recited in claim 13, wherein the first material and the second material are less abrasive than the third material.

18. The method of utilizing a holder for holding a surgical instrument having the physical characteristics of a cautery during a medical operation comprising:
removing a protective sheet covering an adhesive surface disposed on the holder and adhesively attaching the holder to the surgical drape or a portion of the patient's body, said holder further including a surface portion, and a base portion, said adhesive surface disposed on an outer surface of the base portion;
securing a pointed portion of the surgical instrument within the surface portion;
removing and reinserting the surgical instrument in the surface portion throughout the duration of the medical operation;
discarding the holder once the medical operation is completed.

19. The method as recited in claim 18, further including the step of cleaning the pointed portion of the surgical instrument while removing and reinserting the surgical instrument in the surface portion.

20. The method as recited in claim 18, wherein the surface portion comprising a first section and a second section, said first section constructed of a first material and said second section constructed of a second material, wherein said first material is less abrasive than said second material.

21. The method as recited in claim 20, wherein the second section having a V-shaped configuration.

22. The method as recited in claim 21, wherein the second section further including a backing surface, said backing surface comprising a third material, wherein said first material and said second material are less abrasive than said third material.
